(19) United States
(12) Patent Application Publication
(10) Pub. No.: US 2003/0073900 A1
(43) Pub. Date: Apr. 17, 2003

(54) System and method for monitoring the movement of an interventional device within an anatomical site

(76) Inventors: Pranitha Senarith, Temecula, CA (US); Cindy L. Sherman, Temecula, CA (US); Michael D. Whitt, Sunnyvale, CA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 09/977,525

(22) Filed: Oct. 12, 2001

(51) Int. Cl. A61B 5/00; A61M 25/00

(52) U.S. Cl. 600/424; 600/585

(57) ABSTRACT

A magnetometer transducer located on an interventional device positioned within an anatomical site operates in conjunction with a magnet positioned about the anatomical site to provide present-position coordinate data related to the present position of the device and future present-position coordinate data related to future positions of the device as the device is moved about the anatomical site. A processor receives the present-position coordinate data from the transducer and compares the present-position coordinate data to past-position coordinate data stored in a database to determine if the present position is substantially the same as a past position. The processor outputs repeat-position indication data if the present-position data is substantially the same as past-position data. A visual display device displays repeat-position indication data while an audio device produces an audible sound when repeat-position indication data is output.

### BACKGROUND OF THE INVENTION

1. Field of the Invention

The invention relates generally to interventional-device guidance systems and methods and, more particularly, to a system and method for indicating when an interventional device has been manipulated to or through an anatomical location previously passed by the device.

2. Description of the Related Art

One of the most time consuming and inexact interpretations of an electrophysiology (EP) procedure in the heart is the determination of the location of anatomical structure within the heart. One such structure is the coronary sinus. The coronary sinus is the largest cardiac vein which serves as a venous conduit from smaller veins within the myocardium to the right atrium. The coronary sinus extends from an opening for the coronary sinus in the right atrium, along the posterior of the heart to the left side of the heart along the atrioventricular border. When an EP catheter is placed in the coronary sinus, intracardiac electrograms may be obtained from the left atrium as well as the left ventricle if proper contact is made with the designated locations in the heart. In addition, if electrodes are placed on the catheter outside of the coronary sinus, electrograms may be obtained of activity within the right atrium and even from the right ventricle. The location of the electrodes and their size, shape and location on the catheter may vary depending on the needs of the physician and the specific procedures for which the catheter is utilized.

As shown in FIG. 1, a typical human heart contains four chambers, a right and left atrium and right and left ventricle. The right atrium of the heart receives blood returning to the heart through the inferior vena cava (IVC) and superior vena cava (SVC). Adjacent to the opening in the right atrium of the inferior vena cava is the ostium (OS) of the coronary sinus. A tissue fold or primative valve covers the coronary sinus ostium to prevent blood from backflowing into the coronary sinus as it is being pumped out of the right atrium. This coronary sinus ostium is a compliant semi-circular fold comprised of the lining membrane of the atrium. Within the right atrium generally and above the coronary sinus valve specifically is an oval depression called the fossa ovalis (FO). Between the inferior vena cava and the coronary sinus ostium is also the eustaclan ridge (ER). The precise location of each of these elements may vary from patient to patient.

One of the difficulties in performing procedures within the coronary sinus is finding the ostium to the coronary sinus. As earlier stated, the opening or ostium of the coronary sinus is located in the right atrium between the tricuspid valve (TV), the fossa ovalis and the inferior vena cava. Two approaches have been used for the placement of an EP catheter within the coronary sinus, an inferior approach from below the heart and a superior approach from above the heart. In the inferior approach a catheter, especially a steerable catheter, is advanced through the femoral vein into the right atrium. The tip of the catheter is then curved remotely to aim it toward the ostium of the coronary sinus. In the superior approach, a catheter is advanced through either the internal jugular or subclavian vein through the superior vena cava into the right atrium until it is directed toward the coronary sinus.

Gaining access to the ostium of the coronary sinus is a very difficult procedure. In a typical procedure, once a catheter is positioned within the heart, the tip of the catheter is manipulated and moved about within the heart until the physician feels the catheter tip enter the coronary sinus. However, as previously discussed, there are a number of anatomical structures within the right atrium which can be easily confused with the coronary sinus. Accordingly, fluoroscopic imaging is often used to assist in identifying the location of the coronary sinus. Often these particular features of the heart do not show up well on a fluoroscope, thus making the procedure quite difficult and time consuming for the physician.

The physician manipulates the catheter tip under fluoroscopic guidance until the coronary sinus is found. Given the inaccuracy of the fluoroscopic image and the complexity of the heart anatomy, it is not unusual for the physician to manipulate the catheter tip through points previously traversed by the catheter tip. The duplicative nature of catheter guidance necessarily increases the time it takes to locate the coronary sinus and thus the overall time for the EP procedure.

Hence, those skilled in the art have recognized a need for providing a guidance system for an interventional device that provides a indication when the device is being maneuvered in a duplicate fashion within an anatomical site to thereby assist a clinician in guiding the device to a target in a more efficient manner. The invention fulfills these needs and others.

### SUMMARY OF THE INVENTION

Briefly, and in general terms, the invention is directed to system and method for monitoring the movement of an interventional device within an anatomical site and for indicating when an interventional device has been manipulated to or through an anatomical location previously passed by the catheter.

In one aspect, the invention relates to a system for tracking the location of an interventional device within an anatomical site. The system includes a magnetometer system that provides present-position coordinate data related to the present position of the device and future present-position coordinate data related to future positions of the device as the device is moved about the anatomical site. The system also includes a processor that receives the present-position coordinate data from the magnetometer system and processes the present-position coordinate data. The processor outputs repeat-position indication data when the present-position coordinate data is substantially the same as past-position coordinate data stored in a database. The system also includes a sensory indicator that receives the repeat-position indication data from the processor and processes the data to provide a sensory indication.

By providing an indication of when an interventional device is at a point substantially the same as a point the device was previously at, the system lets the clinician know that she is maneuvering the device through a previously traversed area. If the area is away from the target site, e.g., the ostium of the coronary sinus, the indication induces the clinician to move the device from its present position to another position. The absence of an indication upon subsequent movement of the device serves as an indication that the device is not in a previously traversed area and is possibly closer to the target site. Thus the indications, or absence thereof, provided by the system assist clinicians in decreasing the amount of time its takes to locate a target site thereby decreasing the procedure time.

In a detailed facet of the invention, the magnetometer system includes a transducer located on the device and a magnet that defines the origin of the coordinate system of the coordinate data. In another detailed aspect of the invention the processor processes the coordinate data by comparing the present-position coordinate data to past-position coordinate data. In other detailed aspects, the sensory indicator comprises either one or both of a visual display devise that displays repeat-position indication data and an audio device that produces an audible sound as the sensory indication.

In another aspect, the invention relates to a method of tracking the movement of an interventional device within an anatomical site. Position coordinate data for the device relative to an origin is determined and stored as a past position. Upon movement of the device, subsequent position coordinate data for the device relative to the origin is determined. The subsequent position is compared to at least one of the past positions. Repeat-position indication data is provided if the subsequent position is substantially the same as one of the past positions, otherwise the subsequent position coordinate data is stored as a past position.

In a detailed facet of the invention, position coordinate data and subsequent position coordinate data are determined by positioning a magnet on the body to define the origin of the coordinate system and placing a transducer on the device that operates in conjunction with the magnet to provide position coordinate data. In another detailed facet the subsequent position coordinate data is considered to be substantially the same as a past-position coordinate data when the points defined by the coordinate data are within a specified distance of each other.

In another facet, the invention relates to a method of placing an interventional device at a location within a body. The method includes defining the location as coordinate data of a three-dimensional coordinate system having an origin defined by a magnet positioned relative to the body and positioning a transducer on the device. The transducer operates in conjunction with the magnet to provide real-time position coordinate data related to the position of the transducer. The method also includes moving the device within the body and providing a sensory indication when the position coordinate data is substantially the same as the location coordinate data.

These and other aspects and advantages of the invention will become apparent from the following detailed description and the accompanying drawings which illustrate by way of example the features of the invention.

### BRIEF DESCRIPTION OF THE DRAWINGS

FIG. 1 is a diagram of the interior of a heart showing a catheter tip near the ostium of the coronary sinus;

FIG. 2 is a functional block diagram of a system configured in accordance with the invention including a processor adapted to provide an indication through a video display and/or audio system when an interventional device is at or near a previously passed anatomical point, i.e., a duplicate point;

FIG. 3 is an exemplary video display of a path traveled by an interventional device wherein the device has not passed through any duplicate points;

FIG. 4 is an exemplary video display of a path traveled by an interventional device wherein the device has passed through a duplicate point; and

FIG. 5 is a flow chart of an intervention procedure conducted using the system of FIG. 1.

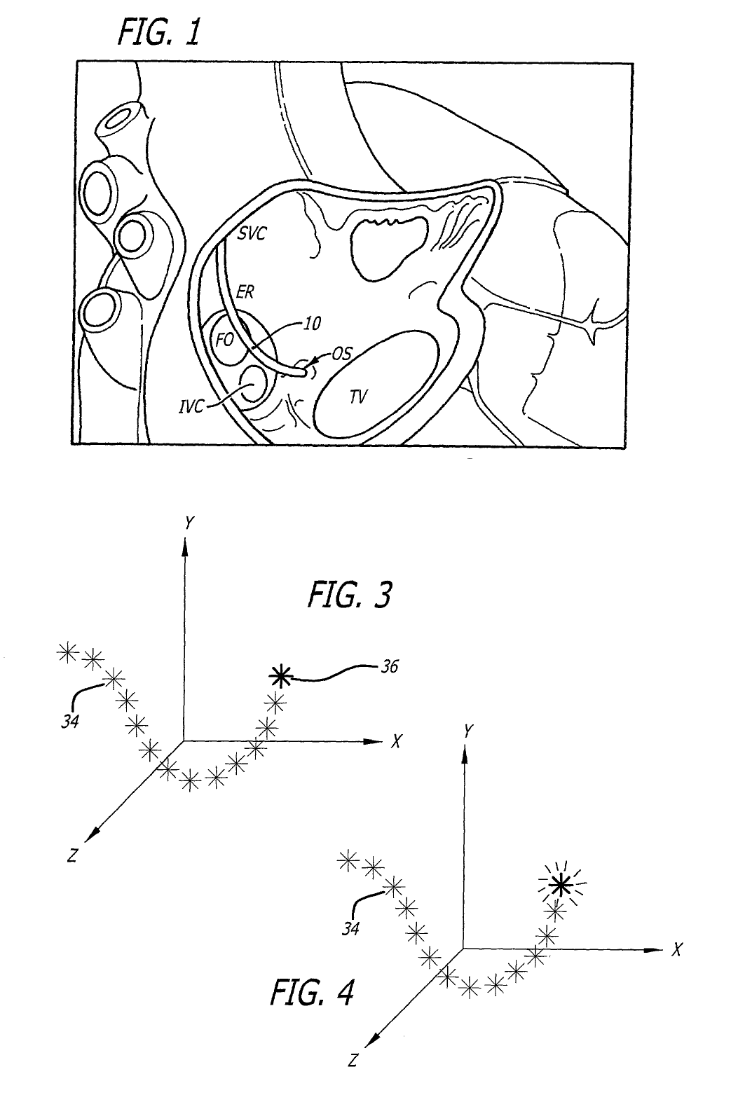

### DETAILED DESCRIPTION OF THE PREFERRED EMBODIMENTS

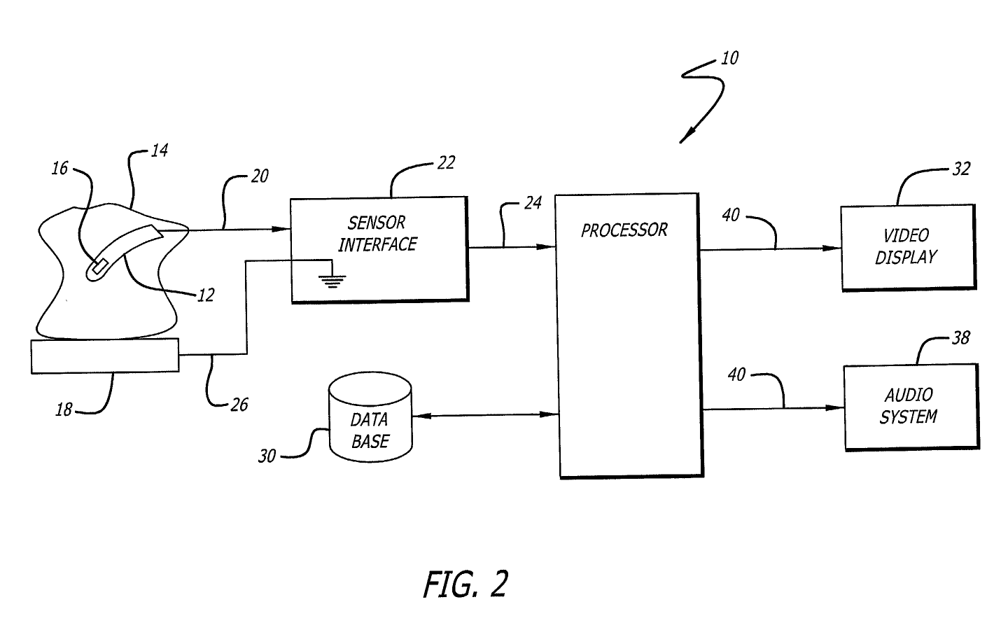

Referring now to the drawings, wherein the reference numerals denote like or corresponding parts throughout the figures, and particularly to FIG. 2, there is shown a system 10 configured in accordance with the invention for monitoring the movement of an interventional device 12 within an anatomical site 14. The system 10 includes a transducer 16 associated with the interventional device 12 and a magnet 18 associated with the anatomical site 14. The transducer 16 is configured to provide three-dimensional coordinate data related to its position relative to the magnet 18. In a preferred embodiment the transducer 16 is a three-axis, solid-state magnetometer sensor such as HMR 2300 manufactured by Honeywell and described in U.S. Pat. No. 5,644,230, the disclosure of which is hereby incorporated by reference.

The transducer 16, operating in conjunction with the magnet 18, provides magnetic signals 20 representative of coordinate data related to the position of the transducer. These magnetic signals 20 are provided in real time, i.e., coordinate data for the transducer 16 is continuously provided as the transducer moves about the anatomical site 14. The magnet 18, typically a grounding magnetic pad located about the exterior of the anatomical site 14, defines the origin of the coordinate system on which the coordinate data is based. The magnetic-signal coordinate data 20 provided by the transducer 16 is input to a sensor interface 22 which converts the magnetic signals from the transducer 16 into analog signals 24 representative of the coordinate data. The sensor interface 22 also provides a ground connection 26 for the magnet 18. The sensor interface 22 may present the analog-signal coordinate data 24 in either Cartesian coordinate form or polar coordinate form. In a preferred embodiment, the sensor interface 22 is a microprocessor configured to operate in conjunction with the previously mention Honeywell magnetometer sensor.

The analog-signal coordinate data 24 generated by the sensor interface 22 is input to a processor 28. Upon receipt of the coordinate data 24, the processor 28 compares the coordinate data, i.e., present-position data, with other coordinate data, i.e., past-position data, stored in a database 30. As used herein, “present-position data” comprises the coordinate data that defines the present position of the transducer 16. “Past-position data” comprises coordinate data that defines one or more points previously traversed by the transducer 16.

If the present-position data defines a point that is within a specified range of a point defined by the past-position data, the processor 28 indicates that the present position of the transducer 16 is the same as one of its past positions. The processor 28 indicates this situation by generating repeat-position indication data, which is described further below. The specified range is dependent on the resolution of the transducer 16. For the Honeywell transducer contemplated for use in the preferred embodiment of the system, the specified range would be a linear distance of approximately 0.5 mm. Accordingly, if the point defined by the x, y, z coordinates of the present-position data is within 0.5 mm of a point defined by the x, y, z coordinates of past-position data, then the present position would be considered the same as a past position. The processor 28 may be programmed to determine the linear distance between points using mathematical formulas well known to those of ordinary skill in the art.

While the processor 28 is configured to process data sufficiently fast to maintain real-time capability, for practical purposes it may not be necessary to compare the present-position data to all past-position data. This is particularly true when the transducer 16 has traveled a substantial distance from its origin point. Therefore, the processor 28 may be configured to compare the present-position data to a limited number of past-position data. For example the processor 28 may compare a specific number of past positions, e.g., the most recent ten or twenty, or only the past positions traversed within a certain amount of time, e.g., the last thirty seconds. By limiting the number of comparisons, system efficiency is enhanced and the system is better able to maintain its real-time capability.

Upon subsequent movement of the transducer 16, new present-position data is provided by the transducer 16 and the old present-position data is either discarded or stored in the database 30 as past-position data. In one embodiment, old present-position data is discarded when the processor 28 determines that the old present-position data is substantially the same as past-position data, otherwise it is stored. This determination may be made in the same manner as previously described with respect to the distance between a present point and a past point. In another embodiment, all old present-position data is stored in the database 30 in order to compile data related to the number of times position coordinate data is repeated. This data provides information related to the number of times duplicate coordinate points are traversed during an intervention. Such data may prove useful in training clinicians and reducing overall procedure time.

The processor 28 converts the analog coordinate data 24 into digital data compatible with a video display 32 for displaying the position of the transducer 16 on the coordinate system in use. With reference to FIG. 3, in one configuration the coordinate system in use is the Cartesian coordinate system, as represented by the x, y and z, axes. The asterisks 34 on the display represent past and present positions of the transducer 16, with the brightest asterisk 36 representing the present position. In order to prevent the display from becoming saturated with asterisks 34, 36, the processor 28 may limit the number of displayed asterisks, for example, by displaying a limited number of past positions, e.g., the most recent ten or twenty, or by displaying only those positions traversed within a certain time frame, e.g., last thirty seconds.

As previously mentioned, when the present position of the transducer 16 is within a certain range of a past position, i.e., when the processor 28 determines that the transducer has traversed a previously traversed point, the processor generates repeat-position indication data. This repeat-position indication data 40 is provided to either one or both of the video display 32 and the audio system 38. With respect to the video display 32, as shown in FIG. 4, the repeat-position indication data 40 causes the asterisk 36 associated with the present-position data to flash. With respect to the audio system 38, the repeat-position indication data 40 causes the audio system speaker to output sound. Both of the visual and auditory responses of the system 10 serve as an indication to the user that the transducer 16 is located at a duplicate coordinate position. The video display and audio system provide the clinician constant monitoring capability to identify duplicated points and provide a method of decreasing the repeated traversal of incorrect points toward attempting to successfully locate a particular anatomical target, such as the coronary sinus

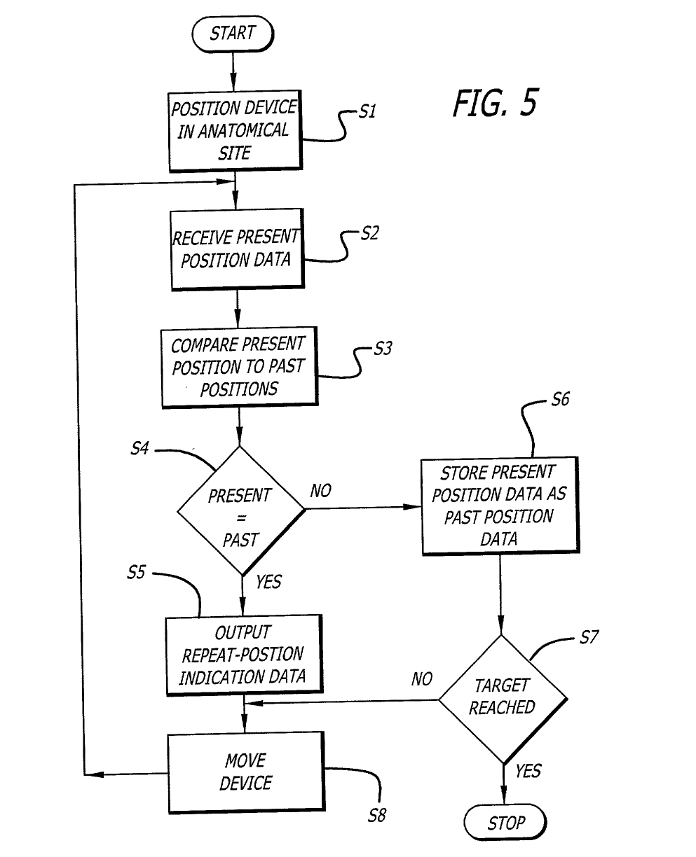

With reference to FIG. 5, in an exemplary use of the system 10 for placing a catheter in the ostium of the coronary sinus of a heart, at step S1, a catheter with a transducer is placed within the heart under fluroscopic guidance. At step S2, the processor receives coordinate data from the sensor interface that define the present position of the transducer. At step S3, the processor compares the present-position data to the past-position data stored in the database and at step S4, determines if the present-position coordinate data is substantially the same as a past-position data. If it is the same, the processor, at step S5 outputs repeat-position indication data to either one or both of the video display and audio system to provide a sensory indication to the user that the catheter is located at a position previously traversed. If the present-position data is not the same as a past-position data then, at step S6, the processor stores the present-position data in the database. At step S7, if the user determines that the catheter has not reached its intended site, e.g., the ostium of the coronary sinus, the user, at step S8 moves the catheter and the process returns to step S2. If the catheter has reached its target then the process ends.

While the preceding description has focused on use of the system for guiding and tracking the position of a catheter within the heart, the invention is in no way limited to such uses and may be applied to other interventional procedures. For example, the system may be used to provide a coordinate tracking system for use in drug delivery, therapeutic agent delivery and monitoring of growth factors on cells. When used for drug delivery, the system allows the user to know exactly where the drug has been delivered such that the user can return to that location to evaluate the condition or to deliver additional drugs.

It will be apparent from the foregoing that while particular forms of the invention have been illustrated and described, various modifications can be made without departing from the spirit and scope of the invention. Accordingly, it is not intended that the invention be limited, except as by the appended claims.

What is claimed is:

1. A system for tracking the location of an interventional device within an anatomical site, said system comprising:
a magnetometer system adapted to provide present-position coordinate data related to the present position of the device and future present-position coordinate data related to future positions of the device as the device is moved about the anatomical site;
a processor communicating with a database having stored therein the coordinate data of a plurality of past positions of the device, the processor adapted to receive present-position coordinate data, process the present-position coordinate data and output repeat-position indication data when present-position coordinate data is substantially the same as one of the past-position coordinate data; and
a sensory indicator adapted to receive the repeat-position indication data from the processor and process the data to provide a sensory indication.

2. The system of claim 1 wherein the magnetometer system comprises a transducer located on the device and a magnet defining the origin of the coordinate system of the coordinate data.

3. The system of claim 2 wherein the magnet comprises a grounding magnetic pad located about the anatomical site.

4. The system of claim 1 wherein the processor is adapted to process the coordinate data by being further adapted to compare the present-position coordinate data to past-position coordinate data.

5. The system of claim 4 wherein the processor is adapted to compare the present-position coordinate data to a select group of past-position coordinate data.

6. The system of claim 5 wherein the select group comprises a select number of past-position coordinate data stored prior to receipt of the present-position coordinate data.

7. The system of claim 5 wherein the select group comprises past-position coordinate data stored within a select time frame prior to receipt of the present-position coordinate data.

8. The system of claim 1 wherein both present- and past-position coordinate data comprise Cartesian coordinate data.

9. The system of claim 1 wherein both present- and past-coordinate data comprise polar coordinate data.

10. The system of claim 1 wherein the sensory indicator comprises a visual display devise adapted to display repeat-position indication data.

11. The system of claim 10 wherein:
the processor is further adapted to output past-position coordinate data to the sensory indicator; and
the sensory indicator is adapted to display past-position data in a manner different from the repeat-position indication data.

12. The system of claim 10 wherein:
the processor is further adapted to output past-position coordinate data and present-position coordinate data to the sensory indicator; and
the sensory indicator is adapted to display present-position coordinate data in a manner different from the past-position data.

13. The system of claim 1 wherein the sensory indicator comprises an audio device adapted to produce an audible sound as the sensory indication.

14. The system of claim 1 wherein the processor is further adapted to compile data related to the number of times repeat-position indication data is output.

15. A method of tracking the movement of an interventional device within an anatomical site, said method comprising:
determining position coordinate data for the device relative to an origin;
storing the position coordinate data as a past position; and
upon movement of the device:
determining subsequent position coordinate data for the device relative to the origin;
comparing the subsequent position to at least one of the past positions; and
providing repeat-position indication data if the subsequent position is substantially the same as one of the past positions, otherwise storing the subsequent position coordinate data as a past position.

16. The method of claim 15 wherein determining position coordinate data and subsequent position coordinate data comprises:
positioning a magnet about the anatomical site to thereby define the origin of the coordinate system; and
placing a transducer on the device, the transducer adapted to operate in conjunction with the magnet to provide position coordinate data.

17. The method of claim 15 wherein subsequent position coordinate data is compared to a select group of past-position coordinate data.

18. The method of claim 17 wherein the select group comprises a select number of past-position coordinate data stored prior to determination of the subsequent position coordinate data.

19. The method of claim 17 wherein the select group comprises past-position coordinate data collected within a select time frame prior to determination of the subsequent position coordinate data.

20. The method of claim 15 wherein the subsequent position coordinate data is substantially the same as a past-position coordinate data when the points defined by the coordinate data are within a specified distance of each other.

21. The method of claim 15 further comprising, in response to repeat-position indication data, providing a visual indication that the subsequent position is substantially the same as one of the past positions.

22. The method of claim 15 further comprising, in response to repeat-position indication data, providing a auditory indication that the subsequent position is substantially the same as one of the past positions.

23. A method of placing an interventional device at a location within an anatomical site, said method comprising:
defining the location as coordinate data of a three-dimensional coordinate system having an origin defined by a magnet positioned relative to the anatomical site;
positioning a transducer on the device, the transducer adapted to operate in conjunction with the magnet to provide real-time position coordinate data related to the position of the transducer;
moving the device within the anatomical site; and
providing a sensory indication when the position coordinate data is substantially the same as the location coordinate data.
